(19) United States
(12) Patent Application Publication
(10) Pub. No.: US 2010/0130863 A1
(43) Pub. Date: May 27, 2010

(54) SYSTEM AND METHOD FOR LOCATION OF ANOMALIES IN A BODY SCAN

(76) Inventor: Kevin M. Kelly, Venice, CA (US)

Correspondence Address: CONNOLLY BOVE LODGE & HUTZ, LLP, P O BOX 2207, WILMINGTON, DE, 19899, US

(21) Appl. No.: 12/626,874

(22) Filed: Nov. 27, 2009

Related U.S. Application Data
(60) Provisional application No. 61/118,436, filed on Nov. 27, 2008.

(51) Int. Cl. A61B 8/14

(52) U.S. Cl. 600/443

(57) ABSTRACT

A body scanning system includes body scan equipment, a marker, and an image processor. The body scan equipment has a scan field and is configured to generate one or more scan images showing features of tissue within a body, with at least part of the body being placed within the scan field. The marker is configured to be disposed at a surface of the body within the scan field and to appear as an image artifact in at least one of the scan images. The image artifact has a profile which distinguishes it from the features of tissue appearing in the scan image(s). The image processor is configured to identify the image artifact in the scan image(s).

### Priority

Priority is claimed to U.S. provisional patent application No. 61/118,436, filed Nov. 27, 2008, the disclosure of which is incorporated herein by reference in its entirety.

### BACKGROUND OF THE INVENTION

1. Field of the Invention

The field of the present invention is systems and methods for locating anomalies in an image or images produced from scans of a body, and particularly for locating anomalies in images resulting from an ultrasound scan of a body.

2. Background

Medical imaging equipment is typically used to identify anomalies within the human body that cannot otherwise be detected by non-surgical methods. Such anomalies might include lesions, tumors, whether benign or malignant, or any other type of structure or growth which is not normally present in healthy body physiology. Many different types of imaging equipment exist which enable scans of part of or the entire body, such as radiographic imaging, Magnetic Resonance Imaging (MRI), tomographical imaging, ultrasound imaging, and the like. Typically, once the body images are obtained, regardless of the imaging technology used, a qualified physician reviews the images to identify any anomalies that may be present within the scanned body tissues. An identified anomaly is then located within the body through use of a physiological reference point, which is a known structure within the body that is easily identified in both the images and during any subsequent invasive surgical procedure. With both the anomaly and the reference point identified, it is possible to have a computer more precisely locate the anomaly within the body by distance and relative position between it and the physiological reference point. This more precise location of the anomaly has been found to be beneficial to physicians when performing a subsequent surgical procedure to treat, biopsy, or remove the identified anomaly.

The drawback to these systems is that the physician is required to view the scan images to identify the physiological reference point. Automation of the identification process is a difficult matter, at best, because any single physiological reference point can have such a wide variation in appearance from person to person. For example, in the context of ultrasound scans of breast tissue, the patient's nipple is frequently used as the physiological reference point when a more precise location of an identified anomaly is desired. However, since different patients have different tissue structures with different densities, the nipples of different patients rarely have the same appearance in scan images of each patient's breast tissue. Thus, before a computer can be used to identify the location of an identified anomaly with respect to the nipple as a physiological reference point, a skilled physician must review the scanned ultrasound images to identify the physiological reference point for the computer. Even though the skilled physician's time is in high demand and very valuable, this step must still be done because of the significant difficulties involved in automating identification of the nipple in the scan images as the physiological reference point. Further, because of the seemingly simple nature of this identification task, many skilled physicians view it as a waste of their valuable time.

### SUMMARY OF THE INVENTION

The present invention is directed toward a system and method for location of anomalies in a body scan. For the system, body scan equipment having a scan field is configured to generate one or more scan images showing features of tissue within a body, with at least part of the body being placed within the scan field. A marker is configured to be disposed at a surface of the body and within the scan field and to appear as an image artifact in at least one of the scan images. The image artifact has a profile which distinguishes it from the features of tissue appearing in the at least one scan image. An image processor is configured to identify the image artifact in at least one of the scan images. The image processor may further be configured to determine the location of an anomaly identified in the scan images relative to the location of the image artifact.

Other options may be incorporated into the body scanning system. As one option, the profile of the image artifact may include at least one of a shape profile and a density profile. As other options, properties of the marker are selected such that the image artifact does not extend into the features of the tissue in the scan images, the marker does not block features of the tissue from showing in the scan images, and/or the marker does not distort features of the tissue in the scan images. Where the body scan equipment utilized is an ultrasound scan equipment, the marker may be a round metal pellet, and it may further be incorporated into a support structure, such as a pad or other aid, which is configured to rest on the body.

The method of locating an anomaly within one or more scan images showing features of tissue within a body starts with placing a marker on a body. At least part of the body is placed within the scan field of body scan equipment. The tissue within the scan field is scanned with the body scan equipment to generate the scan images, such that the marker appears as an image artifact in at least one of the scan images. The image artifact has a profile in the scan image or images which distinguishes it from the features of tissue appearing in the scan images. An image processor is employed to identify the location of the image artifact within the scan images. Thereafter, once the location of a tissue anomaly is identified within the scan images, the image processor is used to determine the spatial location of the tissue anomaly with respect to the location of the image artifact. Options similar to those described above for the system may also be implemented for the method.

Accordingly, an improved system and method for location of anomalies in a body scan are disclosed. Advantages of the improvements will appear from the drawings and the description of the preferred embodiment.

### BRIEF DESCRIPTION OF THE DRAWINGS

In the drawings, wherein like reference numerals refer to similar components:

FIG. 1 schematically illustrates a system for locating anomalies in a body scan;

FIGS. 2A-C illustrate a nipple pad with an integrated marker; and

FIG. 3 is a flowchart describing use of a marker within a scanning system.

### DETAILED DESCRIPTION OF THE PREFERRED EMBODIMENTS

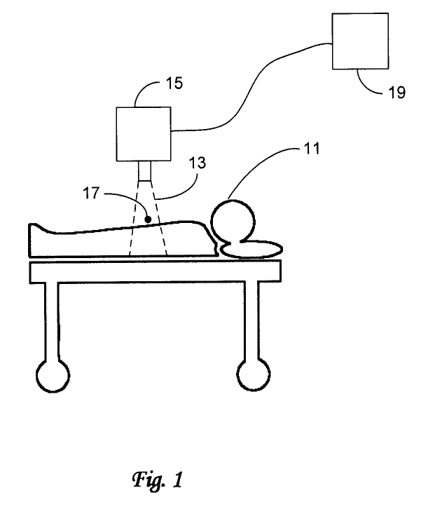

Turning in detail to the drawings, FIG. 1 illustrates a system for locating anomalies in a body scan of a patient 11. The patient 11 is positioned within the scan field 13 of the body scan equipment 13, which may be any type of body scan equipment capable of generating scan images of tissues within the patient's body, the scan images being either digital images or lending themselves to digitization. While there are many different types of such scan equipment, as noted above, the system and method disclosed herein are described employing ultrasound scanning equipment, which is generates a series of scan images showing slices of tissue from the body, or at least part of the body, placed within the scan field. Such equipment is well known to those of skill in the art, and as such the details of the ultrasound scanning equipment is not discussed in detail. U.S. Pat. Nos. 6,524,246 and 6,932,768 describe an ultrasound scanning system which may be used as the body scan equipment. The disclosures of the aforementioned patents are incorporated herein by reference as if set forth in their entirety. The scanning system described therein includes a handheld scan head which is tracked in spatial position and orientation by appropriate tracking devices attached to the handheld scanner. The position and orientation information is associated with each generated scan image for later processing as described below. Alternatively, the position and orientation of the scan head may be controlled by a support arm such that the position and orientation are known and may be thereafter associated with each scan image.

An external marker 17 is placed within the scan field 13, and upon the patient's body at a location near the tissue being scanned, so that it will appear in the images. The marker 17 is preferably placed immediately adjacent the patient's skin, although it may be placed against an outer garment. This marker 17 may be of any appropriate material, size, geometry, density, and the like, and is selected with properties that give it a distinct appearance as an artifact within the scan images. Further, the marker is selected for properties such that for the can technology used, the image artifact left by the marker does not cause shadows, distortions, or otherwise hide important tissue features within that show up within the scan images of the patient's body. Any property of the marker may be changed depending upon the type of scan technology used. For example, properties that may be selected for the marker include type of material, size, geometry, density, and the like. Since the same marker, or at least a marker of the same design, can be used for all patients, the marker will show up as a consistent image artifact in scan images, regardless of patient physiology, so long as the same technology is used for producing the scan images. For an ultrasound scan of breast tissue, the marker may be selected as a small, metal, round pellet which is positioned over the nipple of the patient during the scanning procedure.

The body scan equipment 13 produce the scan images and provides them to an image processor 19, to which it is communicably connected. At the image processor 19, the scan images may be input into an appropriate computer algorithm capable of processing the scan images and readily identifying the image artifact created by the marker. The image processor 19 may be used to automate location of the image artifact within the scan images. Image processing algorithms are well known to those of skill in the art which may be used to process the images and identify the artifact left by the marker. Further, once either a physician, or alternatively the image processor 19, has identified a potential anomaly within the scan images, further automated processing may be performed by the image processor 19 to determine the spatial locational relationship within the scan images, and thus within the patient's body, between the marker artifact and any the identified anomaly. In particular, the image processor 19 may use the scan images, and the known geometrical relationship in and between the various scan images, to determine a more precise location of the anomaly with respect to the image artifact created by the marker. This further processing of the images, once one or more anomalies has been identified, may be performed in the same manner as described in U.S. Pat. No. 6,932,768.

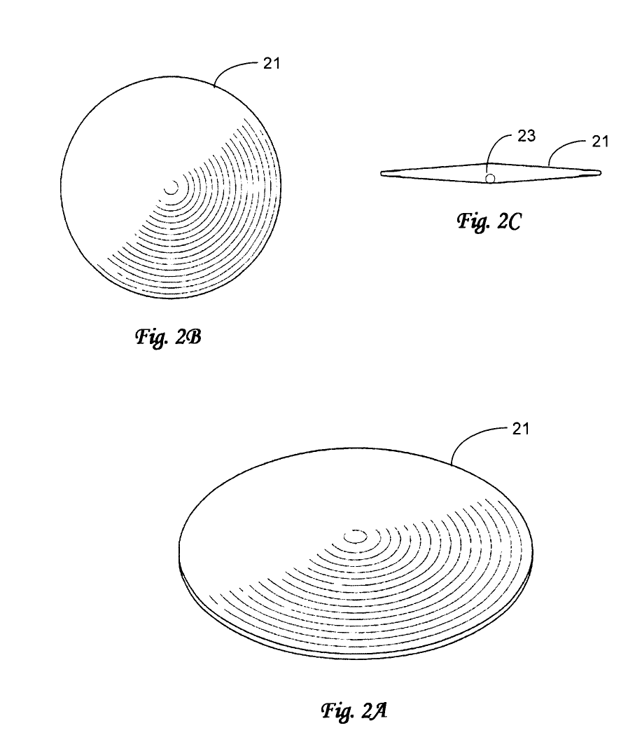

When a pellet, as described above, is used in conjunction with an ultrasound system for breast cancer screening, the pellet may be incorporated into a support structure, such as the nipple pad shown in FIGS. 2A-C. The nipple pad is a lenticular shaped disc 21 and includes a cavity 23 near one side of the disc at the thickest point of the disc. The pellet is placed directly in this cavity 23, such that when the disc 21 is placed over the nipple, with the cavity 23 nearest the tissue, the marker rests almost directly on the tissue, and directly over the patient's nipple. The image artifact created by this marker in the scan images is very distinguishable artifact, and it may be conveniently and readily identifiable by well-known image processing algorithms, such as might be employed by the image processor. Further, because the same type of marker may be used in every ultrasound scan, the automated image processing algorithms may be easily implemented to identify the artifact within the scan images of different patient's, thereby enabling further simplification of the algorithms. Thus, when an anomaly is identified within the scan images, whether through a qualified physician identifying the potential anomaly or through an automated system identifying the anomaly, the image processing algorithm can easily spatially locate the anomaly with respect to the artifact, providing a relative distance and direction of the anomaly with respect to the location of the image artifact. Further, in an ultrasound scan of breast tissue, where the marker is placed directly on top of the nipple, when a subsequent surgical procedure is performed to treat, biopsy, or remove the anomaly, the surgeon may use the nipple as the effective point of reference during the surgical procedure.

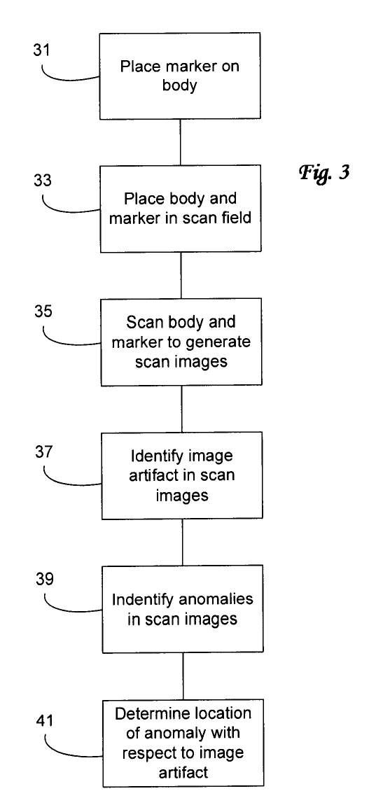

The method of locating an anomaly within a set of scan images is described in FIG. 3. The process starts with placing a marker on the body of a patient undergoing a scan procedure 31. Once the marker is placed, the marker and the body are placed in the scan field 33 of the body scan equipment, and the tissue is scanned 35 to acquire scan images, being sure to include the marker within the field of view of the acquired scan images, processing the images with image processing software to identify the artifact 37 representing the marker in the images, identifying a potential anomaly 39 within the images and using the image processor to determine the spatial location 41 of the anomaly with respect to the position of the marker artifact in the images. In the context of breast tissue scans, the marker is preferably placed directly on the nipple of the patient. In addition, the marker may be incorporated into the nipple pad as described above.

Thus, a system and method for location of anomalies in a body scan are disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A body scanning system comprising:
body scan equipment having a scan field and being configured to generate one or more scan images showing features of tissue within a body, wherein at least part of the body is placed within the scan field;
a marker configured to be disposed at a surface of the body and within the scan field and to appear as an image artifact in at least one of the scan images, wherein the image artifact has a profile which distinguishes the image artifact from the features of tissue appearing in the scan images; and
an image processor configured to identify the image artifact in at least one of the scan images.

2. The system of claim 1, wherein the profile is at least one of a shape profile and a density profile.

3. The system of claim 1, wherein the image artifact does not extend into the features of the tissue in the scan images.

4. The system of claim 1, wherein the marker does not block features of the tissue from showing in the scan images.

5. The system of claim 1, wherein the marker does not distort features of the tissue in the scan images.

6. The system of claim 1, wherein the body scan equipment comprises ultrasound scan equipment.

7. The system of claim 6, wherein the marker comprises a round metal pellet.

8. The system of claim 7, wherein the pellet is incorporated into a support structure configured to rest on the body.

9. The system of claim 1, wherein the image processor is further configured to determine a location of an anomaly identified in the scan images relative to the image artifact.

10. A method of locating an anomaly within one or more scan images showing features of tissue within a body, the method comprising
placing a marker at a surface of the body;
placing the at least part of the body within a scan field of body scan equipment;
scanning at least part of the tissue within the scan field with the body scan equipment to generate the scan images, wherein the marker appears as an image artifact in at least one of the scan images, the image artifact having a profile which distinguishes the image artifact from the features of tissue appearing in the at least one scan image;
identifying a location of the image artifact within the scan images with an image processor;
identifying a location of a tissue anomaly within the scan images; and
determining, using the image processor, a spatial location of the tissue anomaly with respect to the image artifact based upon the identified location of the image artifact and the identified location of the tissue anomaly.

11. The method of claim 10, wherein the profile is at least one of a shape profile and a density profile.

12. The method of claim 10, wherein the image artifact does not extend into the features of the tissue in the scan images.

13. The method of claim 10, wherein the marker does not block features of the tissue from showing in the scan images.

14. The method of claim 10, wherein the marker does not distort features of the tissue in the scan images.

15. The method of claim 10, wherein the body scan equipment comprises ultrasound scan equipment.

16. The method of claim 15, wherein the marker comprises a round metal pellet.

17. The method of claim 16, wherein the pellet is incorporated into a support structure configured to rest on the body.
